(19) United States
(12) Patent Application Publication
(10) Pub. No.: US 2004/0006357 A1
(43) Pub. Date: Jan. 8, 2004

(54) Tweezers having a protective sheathing

(76) Inventor: Fides P. Baldesberger, Lugano (CH)

Correspondence Address: NATH & ASSOCIATES, 1030 15th STREET, 6TH FLOOR, WASHINGTON, DC, 20005, US

(21) Appl. No.: 10/398,446

(22) PCT Filed: Oct. 5, 2001
(86) PCT No.: PCT/CH01/00606

(30) Foreign Application Priority Data
Oct. 25, 2000 (DE) 100 52 957.7

(51) Int. Cl. A61B 17/32

(52) U.S. Cl. 606/167

(57) ABSTRACT

The invention relates to a pair of tweezers (12) with a protective sheathing (11) serving as a packaging; the pair of tweezers has two legs (121, 123) resiliently connected with each other and having ends (122, 124) spaced apart when the pair of tweezers is not in use and which come into contact with each other under the impact of a closing pressure; the protective sheathing (11) consists essentially of a tubular body made of a flexible polymer material, at least partially enclosing said pair of tweezers (12) and having a slit (111, 112) extending in a longitudinal direction and being spread by a pair of tweezers (12) pushed into the protective sheathing (11); the pair of tweezers and the tubular body are dimensioned such that the pair of tweezers is held laterally in the protective sheathing and can be removed from the protective sheathing only by manually further spreading the slit.

A pair of tweezers having a protective sheathing. The invention relates to a pair of tweezers with a protective sheathing which serves as a packaging. Herein below, the assembly of a pair of tweezers and a protective sheathing is also termed a “kit” for short.

“Tweezers” herein are gripping instruments having two legs which are resiliently connected together, the ends of which are spaced apart when the tweezers are not in use and which come into contact with one another under the impact of a manually exerted closing pressure.

Tweezers of this kind are typically used as cosmetic tweezers and have been known for a long time. The prior art is described, for example, in EP-A-0 849 048 which includes a description of the shape of a pair of a novel pair of tweezers which, however, is not critical for the invention. It is to be noted in this respect that the pliers-type tweezers which are also disclosed in the above-mentioned publication are not tweezers within the meaning of the invention, because their legs are not connected resiliently with each other but rather by a pivot in the manner of scissors or pliers.

Frequently, packaging of standard tweezers is relatively costly because, as a rule, packaging containers formed of two parts and/or dimensional adaptations of the sheathing to the product packed therein are needed, notably if the position of the pair of tweezers within the packaging should be fixed. Frequently, a protective cap must be provided at the free ends of the tweezers' tip, for example, if the gripping ends of the pair tweezers are to be protected against being damaged, or if they could cause damage or injury, for instance, when kept and carried in a lady's handbag. As a rule, commercial tweezers' packagings are unsuitable as protective sheathings, and vice versa.

The object of the invention is to provide a pair of tweezers with a packaging which is suitable as a protective sheathing and which avoids these disadvantages. Therefore, the terms “protective sheathing” and “packaging” are synonymous in this context.

It has been found that this object can be achieved according to the invention in that the protective sheathing consists essentially of a tubular structure or body which encloses the pair of tweezers at least in part, is made of a flexible polymer material, and is provided with a slit that extends in longitudinal direction and is spread by a pair of tweezers pushed into the protective sheathing, the pair of tweezers and the tubular body being dimensioned so that the pair of tweezers is held laterally within the protective sheathing, and can be removed only from the protective sheathing by further spreading open the slit.

The invention is in particular suitable for tweezers of a state-of-the art configuration, as described, for example, in EP 99810749.

The closing pressure of the pair of tweezers should generally be at least about 100 grams (g), preferably at least about 150 g, and typically about 200 g. The term “closing pressure” is understood in this connection to indicate the force which—as evidenced by the first contact of the two tips of the pair of tweezers distanced from each other when not is use—is just sufficient to close the pair of tweezers. This force, or the pressure manually exerted for this purpose, can be determined simply by pressing together the pair of tweezers positioned on a balance until the legs close, and determining the pressure which causes the first contact of the tips of the legs.

The protective sheathing according to the invention of the pair of tweezers consists essentially of the above-mentioned tubular body. This means that, although additional elements, e.g. an imprint, a suspension clip or closure elements for the ends of the tubular body or an additional sheathing can be used, this is not normally necessary, for instance for storage, sale or use. The qualification “approximately” before a Figure here generally signifies a permissible variation of ±30%.

A hollow formed body whose length is greater than its thickness and which has a shell whose thickness is typically approximately 5 to approximately 15% of the diameter is considered as “tubular” here. The cross section of the tubular body is preferably round and practically constant over its length. A plastics hose which can be manually compressed and returns approximately to its original shape once the load is removed is a preferred example of a tubular body which is suitable for the invention.

The tubular body consists of a flexible polymer material which is preferably transparent or translucent to such extent that the form and color of the pair of tweezers are visible through the protective sheathing. The term “flexible” generally covers polymer materials which can be manually bent into the form of a hose-like extrudate with a wall thickness in the typical range of 0.5-5 mm at ambient temperature, and essentially return into their original shape after having been bent.

Typical preferred polymer materials are homopolymers and copolymers of monomers, such as alkanes, e.g. ethylene, propylene, vinyl compounds, such as vinyl acetate, vinyl chloride, etc., optionally in compatible admixture with each other, as well as polyamides, polyesters, modified polymers based on styrene, butadiene, acrylonitrile, cellulose derivatives, etc., as are known to those experienced in the packaging art, and optionally may contain conventional plasticizers, which, in turn, may be monomeric or polymeric substances. It is also possible to use all other additives which are conventionally used with such polymer materials, such as stabilizers, as long as they do not cause the material to become opaque or non-transparent to transmission of light.

The tubular body is provided with a longitudinal slit, i.e. a longitudinal cut through the shell wall of the tubular body or hose. The slit or cut preferably extends approximately parallel to the axis and across the entire length of the tubular body or hose, respectively, which, in turn, has a typical length at least about equal to that of the pair of tweezers.

The dimensions of the pair of tweezers and of the longitudinally slit tubular body constituting the protective sheathing for the former are such that, upon pushing a pair of tweezers into the tubular body the slit is spread, preferably such that the spread is widest approximately at the centre of the longitudinal dimension of the protective sheathing, this width generally not exceeding approximately 20% and preferably not 10% of the length of the protective sheathing and extending from one end of the protective sheathing to its other end, i.e. the slit extending over the entire length of the protective sheathing is not spread at both ends of the latter.

The form of the cross section of the inner space of the protective sheathing is preferably adapted to the form and dimensions of the tweezers such that the pair of tweezers presses resiliently against the protective sheathing and in so doing spreads the slit to an extent such that the pair of tweezers is essentially secured against unintentional displacement in the longitudinal direction. As the protective sheathing is preferably somewhat longer than the pair of tweezers, the ends thereof are enclosed when the pair of tweezers is disposed approximately in the middle of the protective sheathing and does not require a protective cap.

Due to the flexible nature of the longitudinally slit protective sheathing according to the invention, the pair of tweezers can be effortlessly removed by the user if the slit is opened further by bending apart the wall parts adjoining the slit in the region of the original spread. Once the pair of tweezers has been removed the slit closes, again due to the soft-elastic nature of the protective sheathing. In order to re-introduce the pair of tweezers into the protective sheathing, e.g. after use, the pair of tweezers is pushed into an open end of the protective sheathing at least to the extent that the gripping ends of the pair of tweezers are protected by the protective sheathing.

A preferred but not restrictive example of a kit according to the invention is represented in the accompanying drawings and described in detail below. Same reference numerals denote same parts in the figures of the drawings, in which:

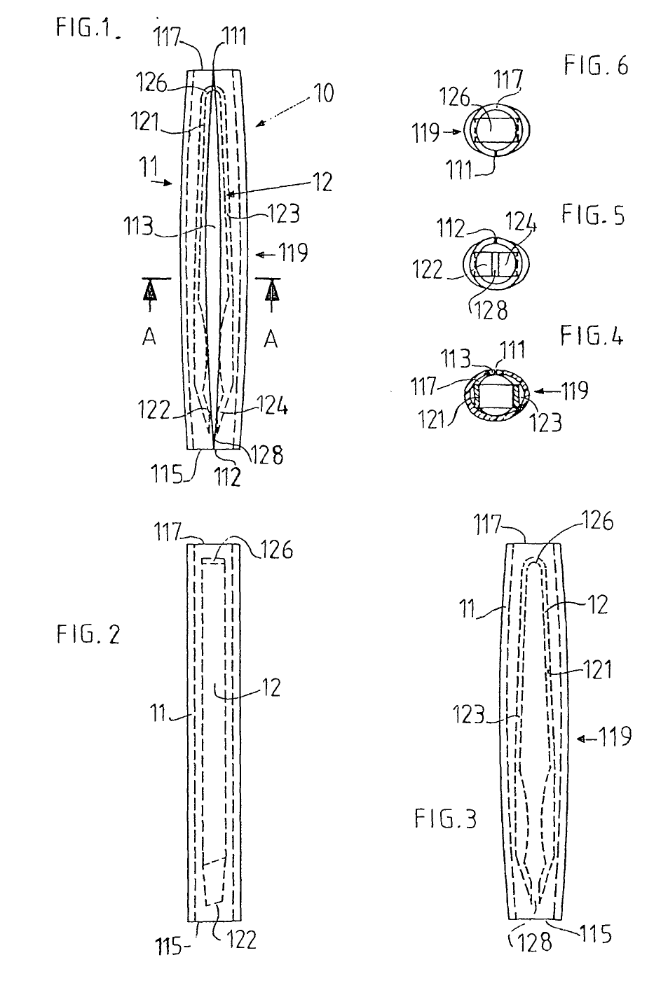

FIG. 1 shows a kit according to the invention with a pair of tweezers and a protective sheathing in a front view, i.e. with the slit positioned at the front;

FIG. 2 shows the kit of FIG. 1 in a side view;

FIG. 3 shows the kit of FIG. 1 in a view from the rear;

FIG. 4 is an enlarged cross section of FIG. 1 along line A-A;

FIG. 5 shows the kit of FIG. 1 in a view from below; and

FIG. 6 shows the kit of FIG. 1 in a view from above.

In detail, FIG. 1 shows kit 10 consisting of protective sheathing 11 and a pair of tweezers 12, e.g. a pair of light metal tweezers of the type described in PCT/CH00/00441 or a pair of standard tweezers made of steel. Protective sheathing 11 consists of a flexible polymer material, e.g. an extruded hose of high-pressure polyethylene with a wall thickness of typically approximately 1-2 mm. Over its entire length, sheathing 11 is provided with an approximately straight longitudinal slit 111, 112, which extends from top end 117 of sheathing 11 to bottom end 115 thereof.

The two legs 121, 123 of the pair of tweezers are resiliently connected together at their top end by a bend 126 and cause a spring pressure corresponding essentially to the closing pressure of the pair of tweezers 12 of typically about 200 g. In other words, a pressure of approximately 200 g is required to bring the two free ends 122, 124 of the pair of tweezers 12 into first contact with each other. As shown in the enlarged cross section in FIG. 4 along line A-A of FIG. 1, the dimensions of sheathing 11 and of the pair of tweezers 12 are such that the pair of tweezers pushes against the sheathing and the spring force of the pair of tweezers 12 spreads slit 111, 112 as represented at 113. This spread 113 corresponds to the outer curvature 119 of sheathing 11.

The flexible polymer material of sheathing 11 is expediently selected such that—in the shape of longitudinally slit sheathing 11—it has sufficient dimensional stability to compensate for the spring pressure of the pair of tweezers 12 to an extent such that the free ends 122, 124 thereof together form a gap 128 (FIG. 5) which is slightly smaller than the normal gap between the free ends of the pair of tweezers 12, i.e. without the effect of a compressive force acting against the spring force of the pair of tweezers. The choice of suitable polymer materials for the sheathing and selection of elasticity properties of the sheathing and the tweezers are technical parameters which can be optimised on the basis of a few simple tests.

Slit sheathing 11 of a kit 10 according to the invention should, generally speaking, exert on the pair of tweezers 12 a compressive force which is not significantly greater and preferably slightly less than the spring force of the pair of tweezers 12, while the spring force of the pair of tweezers 12 should be sufficiently high to spread sheathing 11.

It should, however, be emphasised that it is not necessary to measure these forces, and such forces can be estimated on the basis of a simple criterion: The slit sheathing 11 should generally be spread by a pair of tweezers 12 located therein by not more than approximately 20% of its length and preferably by approximately 10% of its length, because this provides for an advantageous property balance between sufficiently easy removal and sufficiently firm retention of the pair of tweezers 12 in sheathing 11. As is apparent from FIGS. 2 and 3, the aim is to spread sheathing 11 by means of the pair of tweezers 12 only in the lateral direction, i.e. only the rear view of FIG. 3 shows the bulging 119 on both sides which is indicated in FIG. 1, while the walls of sheathing 11 extend substantially without widening in the side view of FIG. 2.

It is understood that various modifications are possible for persons skilled in the art within the scope of the invention. For example, the material of the sheathing may be glass-clear and colorless if the color of the tweezers is to be seen unimpaired from outside. If the pair of tweezers is made of a light metal or steel, it may be metallically silver; however it may also exhibit a color, e.g. through varnishing, coloring an electrically produced oxide layer, vapor deposition, or electroplating. The sheathing and/or the pair of tweezers may comprise patterns, prints, decorations, fastening means and the like, and it resides within the scope of the invention for the pair of tweezers to project out of the sheathing at one end and/or for at least one end of the sheathing to be provided with a closure, e.g. a cap. However the fact that sharp or pointed ends of the pair of tweezers do not require any special protective caps because they can easily be protected by retention in the sheathing, is an advantage of the invention. Inscriptions or labels, e.g. as instructions for use and/or to indicate a price, can easily be provided through a releasable connection with the closed end of the pair of tweezers, e.g. with a retaining clip drawn through the pair of tweezers near its closed end.

Further, it is possible to combine a plurality of pairs of tweezers according to the invention with protective sheathings, for instance for exhibition or sales purposes, to form a coherent elongate web which can easily be separated into its components, i.e. a single pair of tweezers with its protective sheathing.

1. A pair of tweezers (12) with a protective sheathing (11) serving as a packaging, wherein said pair of tweezers has two legs (121, 123) resiliently connected with each other and having ends (122, 124) which are spaced apart when said pair of tweezers is not in use and which come into contact with each other under the impact of a closing pressure, characterised in that said protective sheathing (11) consists essentially of a tubular body made of a flexible polymer material at least partially enclosing said pair of tweezers (12) and having a slit (111, 112) extending in a longitudinal direction and being spread by a pair of tweezers (12) pushed into said protective sheathing (11), and wherein said pair of tweezers and said tubular body are dimensioned such that said pair of tweezers is held laterally in said protective sheathing and can be removed from said protective sheathing only by manually spreading further said slit.

2. The pair of tweezers with said protective sheathing according to claim 1, characterised in that said closing pressure of said pair of tweezers is at least about 100 grams, said slit extending substantially across the total length of said tubular body and being spread by said pair of tweezers by not more than about 20% of its length and preferably not more than about 10% of its length.

3. The pair of tweezers with said protective sheathing according to claims 1 or 2, characterised in that said polymer material is transparent or translucent such that shape and color of said pair of tweezers are visible through said protective sheathing.

4. The pair of tweezers with said protective sheathing according to any of claims 1-3, characterised in that said tubular body has a round inside cross section which is substantially constant over its length and a dimensional elasticity such that said slit remains essentially closed when no pair of tweezers is pushed into said body while said slit is spread only in a portion thereof when said pair of tweezers is pushed into said body.

5. The pair of tweezers with said protective sheathing according to any of claims 1-4, characterised in that said tubular body has a length at least approximately equal to the length of said pair of tweezers and, preferably, is somewhat longer so as to surround both of said ends of said pair of tweezers.

6. The pair of tweezers with said protective sheathing according to any of claims 1-5, characterised in that a marking or label is separably connected to the top end (126) of said pair of tweezers.

7. (new) A kit comprising a pair of tweezers and a protective sheathing serving as a packaging for said pair of tweezers, wherein said pair of tweezers has two legs resiliently connected with each other and having ends which are spaced apart when said pair of tweezers is not in use, and which come into contact with each other under impact of a closing pressure, wherein said protective sheathing consists essentially of a tubular body made of a flexible polymer material, said tubular body at least partially enclosing said pair of tweezers and having a slit extending in a longitudinal direction and being spread by said pair of tweezers when pushed into said protective sheathing, and wherein said pair of tweezers and said tubular body are dimensioned such that said pair of tweezers is held in said protective sheathing but can be removed from said protective sheathing when said slit is spread further.

8. (new) The kit of claim 7, wherein said pair of tweezers requires a closing pressure of at least about 100 grams, said slit extending substantially across the total length of said tubular body and being spread by said pair of tweezers by not more than about 20% of its length.

9. (new) The kit of claim 7, wherein said pair of tweezers requires a closing pressure of at least about 150 grams, said slit extending substantially across the total length of said tubular body and being spread by said pair of tweezers by not more than about 10% of its length.

10. (new) The kit of claim 7, wherein said polymer material is transparent or translucent such that shape and color of said pair of tweezers are visible through said protective sheathing.

11. (new) The kit of claim 7, wherein said tubular body has a round inner cross section which is substantially constant over its length, and a dimensional elasticity such that said slit is essentially closed when said pair of tweezers removed from said tubular body, while a portion of said slit is spread when said pair of tweezers is pushed into said tubular body.

12. (new) The kit of claim 7, wherein said tubular body has a length at least approximately equal to the length of said pair of tweezers.

13. (new) The kit of claim 7, wherein said tubular body has a length greater than said pair of tweezers.

14. (new) The kit of claim 7, wherein a marking means is separably connected to a top end of said pair of tweezers.
